(19) United States
(12) Patent Application Publication
(10) Pub. No.: US 2025/0041107 A1
(43) Pub. Date: Feb. 6, 2025

(54) Weighted Gel Blanket for Targeted Temperature Management Systems

(71) Applicant: C. R. Bard, Inc., Franklin Lakes, NJ (US)

(72) Inventors: Sean E. Walker, Platteville, CO (US); Rebecca D. Salisbury, Lafayette, CO (US); Madeline Stich, Thornton, CO (US); Gabriel A. Johnston, Broomfield, CO (US); Michael R. Hoglund, Windsor, CO (US)

(21) Appl. No.: 18/715,504

(22) PCT Filed: Dec. 23, 2021
(86) PCT No.: PCT/US2021/065144

§ 371 Date: May 31, 2024

(51) Int. Cl. A61F 7/00; A61F 7/02

(52) U.S. Cl. CPC A61F 7/0097; A61F 7/0085; A61F 2007/0054; A61F 2007/0219; A61F 2007/0222; A61F 2007/0253; A61F 2007/0268

(57) ABSTRACT

Disclosed herein a blanket apparatus and systems and methods pertaining to the same. The blanket includes a body portion formed of fabric, wherein the fabric is elastomer or elastomer-based and a first pocket within the body portion and surrounded by the fabric, wherein the first pocket is an enclosed area filled with an insulative material, and wherein the blanket is configured to be placed on at least a first thermal pad of a targeted temperature management (TTM) system, wherein the first pocket is configured to provide a weight on top of the first thermal pad. The insulative material may be an insulative gel, paste or granular material. In some instances, a length of the first pocket is substantially an entirety of a length of the blanket. Additionally, in some instances, a width of the first pocket is substantially an entirety of a width of the blanket.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g., water) through one or more thermal contact pads coupled to a patient to affect surface-to-surface thermal energy exchange with the patient. In general, TTM systems comprise a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001, and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

Currently, many TTM systems utilize thermal contact pads that include an adhesive backing in order hold the thermal contact pad on a particular region of a patient body. However, adhesive-backed thermal contact pads may cause skin damage, especially during patient swelling. Alternative current TTM systems that use non-adhesive-backed thermal contact pads often require additional components (e.g., wraps) to hold the thermal contact pads in place. For example, wraps having adjustable straps utilizing VELCRO® or other latching mechanisms are often used to maintain placement of a thermal contact pad. However, such wraps typically require the clinician move the patient to install the wrap (e.g., sit the patient upright). Such movement is potentially damaging to patient, especially those suffering from traumatic brain injuries.

Thus, what is needed is a system and apparatus for maintaining placement of a thermal contact pad that does not require adhesion to the patient body and also does not require movement of the patient body to install.

### SUMMARY OF THE INVENTION

Briefly summarized, disclosed herein is a blanket comprising a body portion formed of fabric, wherein the fabric is elastomer or elastomer-based and a first pocket within the body portion and surrounded by the fabric, wherein the first pocket is an enclosed area filled with an insulative material, and wherein the blanket is configured to be placed on at least a first thermal pad of a targeted temperature management (TTM) system, wherein the first pocket is configured to provide a weight on top of the first thermal pad. In some embodiments, the insulative material is an insulative gel.

In some embodiments, a length of the first pocket is substantially an entirety of a length of the blanket. In some embodiments, a width of the first pocket is substantially an entirety of a width of the blanket. In some embodiments, the blanket includes a plurality of pockets within the body portion, wherein the plurality of pockets includes the first pocket and wherein each of the plurality of pockets is surrounded by the fabric, and is an enclosed area filled with the insulative material. In some embodiments, each of the plurality of pockets has a length that is substantially an entirety of a length of the blanket, and the plurality of pockets are aligned in a side-by-side configuration.

In some embodiments, each of the plurality of pockets has a width that is substantially an entirety of a width of the blanket, and the plurality of pockets are aligned in a side-by-side configuration. In some embodiments, the blanket includes a coupling component configured to couple with the first thermal pad. In some embodiments, the coupling component is one of an adhesive patch, a snap fastener component, a hook and loop fastener component, or a magnetic component. In some embodiments, when the coupling component is any of the snap fastener component, the hook and loop fastener component, or the magnetic component, the coupling component is configured to couple with a reciprocal coupling component of the first thermal pad.

Also disclosed herein is a targeted temperature management (TTM) system, comprising a TTM module configured to provide a TTM fluid, a thermal pad configured to receive the TTM fluid from the TTM module to facilitate thermal energy transfer between the TTM fluid and a patient, a fluid delivery line (FDL) extending between the TTM module and the thermal pad, the FDL configured to provide TTM fluid flow between the TTM module and the thermal pad, and a blanket comprising a body portion formed of fabric, wherein the fabric is elastomer or elastomer-based, and a first pocket within the body portion and surrounded by the fabric, wherein the first pocket is an enclosed area filled with an insulative material, and wherein the blanket is configured to be placed on at least the thermal pad of a targeted temperature management (TTM) system, wherein the first pocket is configured to provide a weight on top of the thermal pad.

Additionally, disclosed herein is a method of using a targeted temperature management (TTM) system, comprising providing a TTM system that includes a TTM module configured to provide a TTM fluid, a thermal pad configured to receive the TTM fluid from the TTM module to facilitate thermal energy transfer between the TTM fluid and a patient, a fluid delivery line (FDL) extending between the TTM module and the thermal pad, the FDL configured to provide TTM fluid flow between the TTM module and the thermal pad, and a blanket that includes a body portion formed of fabric, wherein the fabric is elastomer or elastomer-based, and a first pocket within the body portion and surrounded by the fabric, wherein the first pocket is an enclosed area filled with an insulative material, and wherein the blanket is configured to be placed on at least the thermal pad of a targeted temperature management (TTM) system, wherein the first pocket is configured to provide a weight on top of the thermal pad, applying the thermal pad to the patient, applying the blanket to the patient on top of the thermal pad, delivering TTM fluid from the TTM module to the thermal pad, and analyzing an efficacy a TTM procedure being performed by TTM system based on the temperature data, wherein the TTM procedure includes providing the TTM fluid. The method may further include the operation of coupling the coupling component of the blanket with the reciprocal coupling component of the thermal pad.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient and a targeted temperature management (TTM) system for cooling or warming the patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4A is a top view of a portion of the thermal contact pad of FIG. 1, in accordance with some embodiments.

FIG. 4B is a cross-sectional side view of the portion the thermal contact pad of FIG. 4A, in accordance with some embodiments.

FIG. 5A is an illustration of blanket having matter-filled pockets disposed throughout the blanket, in accordance with some embodiments.

FIG. 5B provides a cross-sectional view of the blanket of FIG. 5A, in accordance with some embodiments.

FIG. 6A is an illustration of a blanket having gel packets disposed as strips along the length of the blanket, in accordance with some embodiments.

FIG. 6B is an illustration of a blanket having gel pockets disposed as strips along the width of the blanket, in accordance with some embodiments.

FIG. 7 is an illustration of a blanket having gel pockets disposed in the formation of a human body, in accordance with some embodiments.

FIG. 8A is an illustration of a blanket of any of FIGS. 5-7 placed on a patient body showing how the blanket contours to the patient body, in accordance with some embodiments.

FIG. 8B provides a cross-sectional view of the blanket of FIG. 8A, in accordance with some embodiments.

FIG. 9A is an illustration of a blanket having gel pockets and fixedly coupled to a thermal contact pad through hook and loop fasteners, in accordance with some embodiments.

FIG. 9B provides a cross-sectional view of the blanket of FIG. 9A, in accordance with some embodiments.

FIG. 10A is an illustration of a blanket having gel pockets and magnetically coupled to a thermal contact pad, in accordance with some embodiments.

FIG. 10B provides a cross-sectional view of the blanket of FIG. 10A, in accordance with some embodiments.

FIG. 11A is an illustration of a blanket having gel pockets and coupled to a thermal contact pad through snap fasteners, in accordance with some embodiments.

FIG. 11B provides a cross-sectional view of the blanket of FIG. 11A, in accordance with some embodiments.

FIG. 12 is an illustration of a blanket having gel pockets and adhesively coupled to a thermal contact pad, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

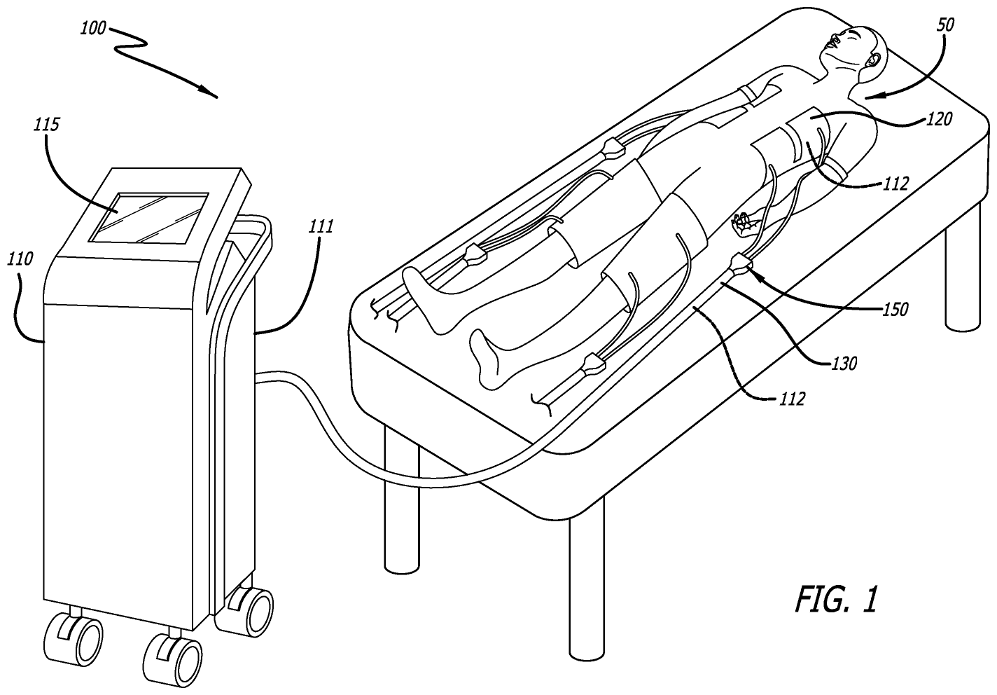

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 comprises a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120. The FDL includes two conduits to facilitate delivery flow of TTM fluid 112 from the TTM module 110 to the pad 120 and return flow TTM fluid 112 from the pad 120 to the TTM module 110. In some embodiments, the two conduits may be attached to each other along a portion of a length of the FDL.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 along a TTM fluid flow path including within the pad 120 to facilitate thermal energy exchange with the patient 50. During the TTM therapy, the TTM module 110 may continually control the temperature of the TTM fluid 112 toward a target TTM temperature.

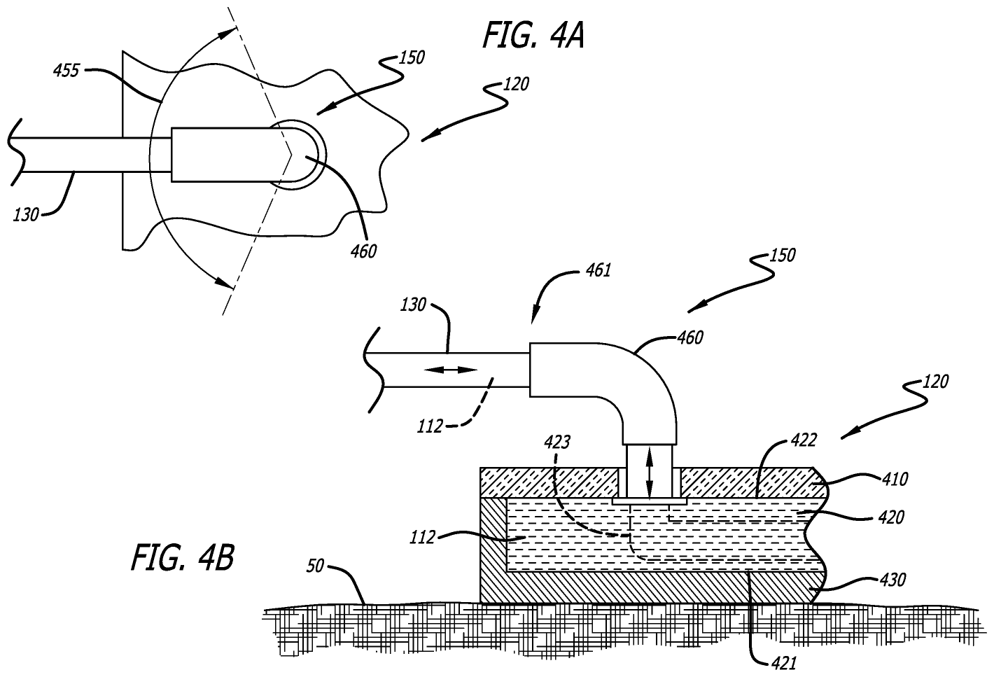

The TTM system 100 may include a connector system 150 to couple the FDL 130 to the pad 120. In some embodiments, the connector system 150 may couple a single fluid conduit of the FDL to the pad 120. Hence, the connection between the FDL 130 and the pad 120 may comprise more than one connector system 150 to couple more than one fluid conduit to the pad 120. The connector system 150 is further described below in FIGS. 4A and 4B.

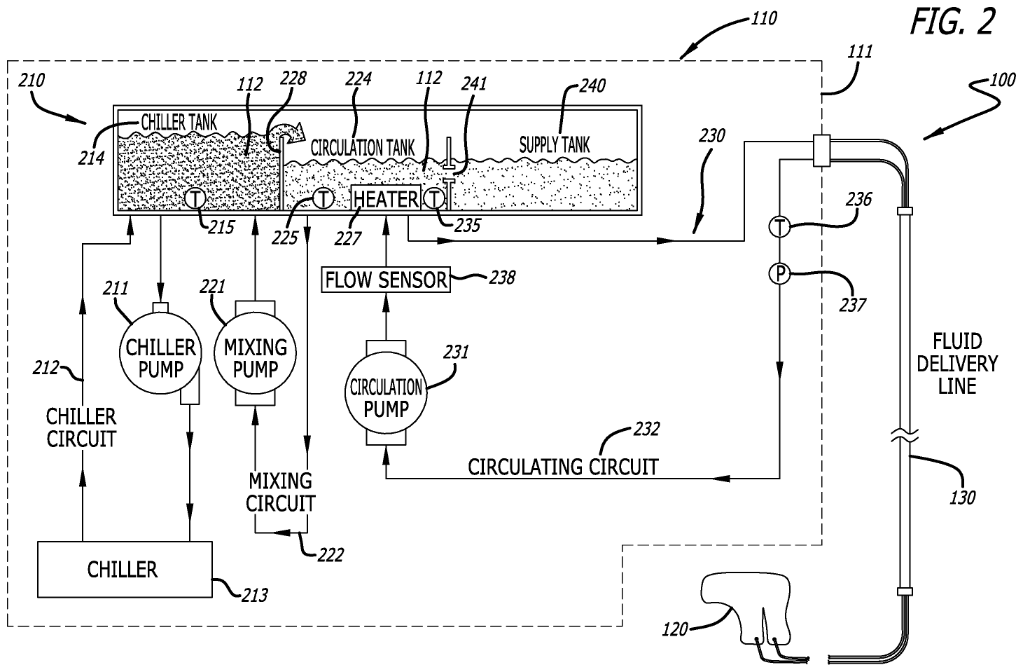

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

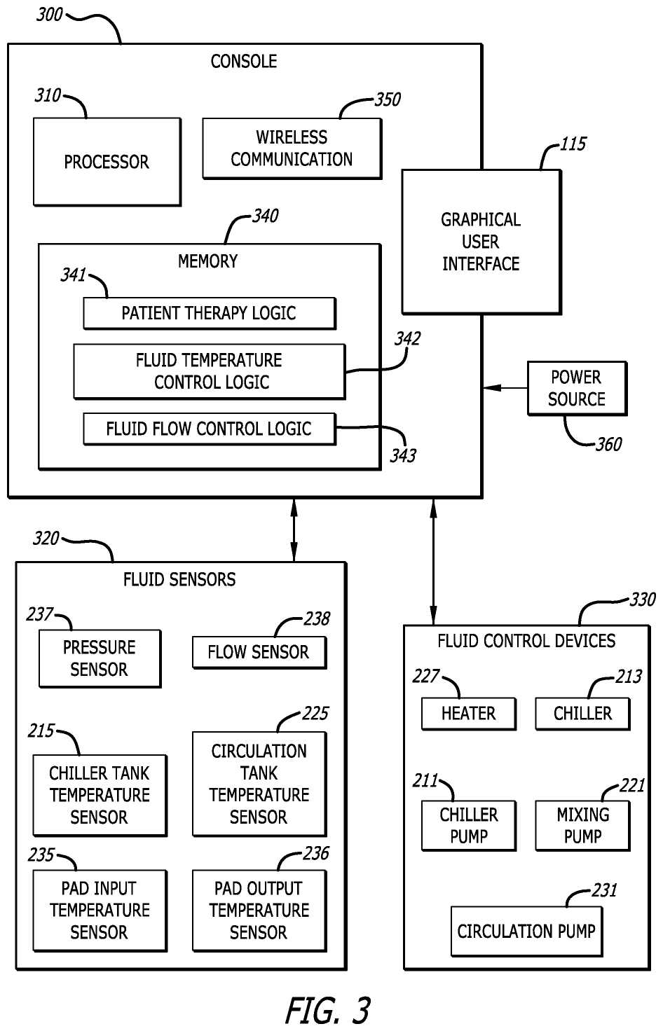

The temperature control subsystem 210 may include a chiller pump 211 to pump (recirculate) TTM fluid 112 through a chiller circuit 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) May 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 comprises a circulation pump 213 to pull TTM fluid 112 from the circulation tank 224 and through a circulating circuit 232 that includes the fluid delivery line 130 and the pad 120 located upstream of the circulation pump 213. The circulating circuit 232 also includes a pressure sensor 237 to represent a pressure of the TTM fluid 112 within the pad 120. The circulating circuit 232 includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation than) the pad 120 so that a pressure within the pad 120 is less than atmospheric pressure (i.e., negative) when TTM fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module 110 includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 and/or a thermal energy exchange rate which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with the predefined target temperature. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224 by mixing TTM fluid 112 from the chiller tank 214 with TTM fluid 112 within circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may include or be couple do wireless communication module 350 to facilitate wireless communication with external devices. A power source 360 provides electrical power to the console 300.

FIG. 4A shows a top view of a portion of the thermal contact pad 120 including the connector system 150 and the FDL 130 extending away from the connector system 150, in accordance with some embodiments. As illustrated, the connector system 150 may provide for a rotatable connection between the FDL 130 and the pad 120. The rotatable connection may provide for the FDL 130 to swivel through an angle 455 ranging up to about 90 degrees, 180 degrees, or 360 degrees.

FIG. 4B shows a cross-sectional side view of an inlet or an outlet of the thermal contact pad 120 of FIG. 1 in contact with the patient 50, in accordance with some embodiments. The pad 120 may comprise multiple layers to provide multiple functions of the pad 120. A fluid containing layer 420 is fluidly coupled to the FDL 130 via the connector system 150 to facilitate circulation of the TTM fluid 112 within the fluid containing layer 420. The fluid containing layer 420 having TTM fluid 112 circulating therein defines a heat sink or a heat source for the patient 50 in accordance with a temperature of the TTM fluid 112.

The pad 120 may include a thermal conduction layer 430 disposed between the fluid containing layer 420 and the patient 50. The thermal conduction layer 430 is configured to facilitate thermal energy transfer between the fluid containing layer 420 and the patient 50. The thermal conduction layer 430 may be attached to the thermal conduction layer 430 along a bottom surface 421 of the fluid containing layer 420. The thermal conduction layer 430 may be conformable to provide for intimate contact with the patient 50. In other words, thermal conduction layer 430 may conform to a contour of the patient 50 to inhibit the presence space or air pockets between the thermal conduction layer 430 and the patient 50.

The pad 120 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the FDL 130 with the fluid containing layer 420.

The connector system 150 may include an elbow 460 to change the direction of FDL 130 extending away from the connector system 150. As shown, the direction of FDL 130 is shifted from a direction perpendicular to the pad 120 to a direction that is substantially parallel to the pad 120. The elbow 460 also establishes an orientation of a distal portion 461 of the FDL 130 to be substantially parallel to the pad 120 and/or the fluid containing layer 420.

In some embodiments, the opening 411 illustrates an inlet port to which the FDL 130 couples such that the TTM fluid 112 may enter into the fluid containing layer 420 and flow freely in a direction as dictated by the negative pressure within the pad 120 resulting from operation of the circulation pump 213. However, in other embodiments, the fluid containing layer 420 may include one or more internal flow paths (illustrated via dashed lines 423) such that the TTM fluid 112 may flow through the internal flow path(s) in a controlled manner in the as dictated by the negative pressure resulting from operation of the circulation pump 213. In some embodiments, e.g., in which FIG. 4B illustrates an inlet port, the TTM fluid 112 exits the pad 120, and specifically the fluid containing layer 420 via an outlet port (not shown) that may resemble the configuration of the as shown in FIG. 4B.

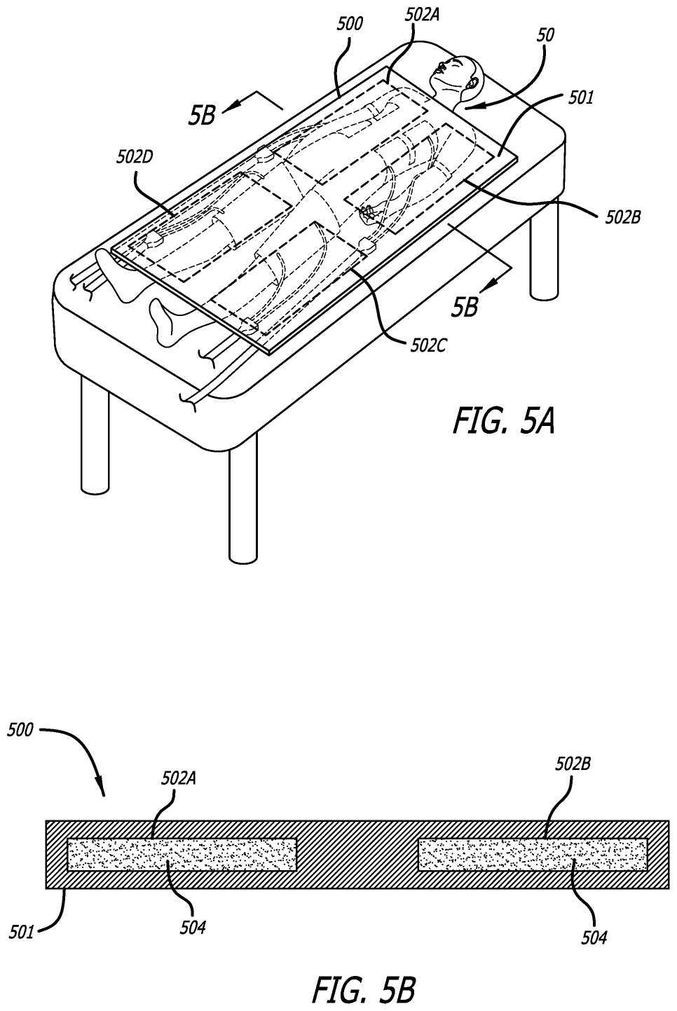

FIG. 5A is an illustration of blanket having matter-filled pockets (e.g., cavities or voids) disposed throughout the blanket, in accordance with some embodiments. In some embodiments, the blanket 500 has a body portion 501 that is formed of elastomer (e.g., an elastomeric blanket) that includes one or more pockets that are filled with an insulative matter. Examples of the insulative matter may include gel material, a high viscosity granular material (e.g., sand, granular fill insultation such as perlite, vermiculite or polystyrene in any of beads, pebbles or pellets), a high viscosity paste material, etc. For purposes of clarity, the term “gel pocket” will be used hereinafter to collectively refer to a pocket filled with an insulative matter as described above. Further, embodiments of blankets disclosed herein will be discussed referencing “gel pocket(s)” and “gel”; however, the pockets may be filled with any of the materials discussed above where the discussion is intended to be illustrative and apply to the various pocket configurations and insulative fill matters.

As is shown in FIG. 5A, the blanket 500 is configured to be placed on top of the thermal contact pads (pads) 120. Due to the elastomeric fabric from which the blanket is formed and conformable nature of the gel 504 filling the gel pockets 502A-502D (individually and collectively “502”), the blanket 500 conforms to the contours of the pads 120 and patient 50 thereby holding the pads 120 in place (e.g., in a stationary position) and causing intimate contact with the patient 50.

As the gel 504 is contained within the gel pockets 502, the gel 504 is generally contained within strategic locations to ensure intimate contact with the patient 50. For instance, gel pockets 502A-502B are shown to be disposed on a first portion of the blanket 500 such that when the blanket 500 is placed on the patient 50, the gel pockets 502A-502B are disposed on top of the pads 120 placed on the chest of the patient 50 and the gel pockets 502C-502D are disposed on top of the pads 120 placed on the legs of the patient 50. As will be apparent from the discussion below and with reference to FIGS. 5A-11, the placement of the gel pads 502 in FIG. 5A is merely one illustrative embodiment and is not intended to be limiting.

FIG. 5B provides a cross-sectional view of the blanket 500, in accordance with some embodiments. FIG. 5B illustrates a cross-sectional view of two gel pockets 502A-502B, where each is filled with the gel 504 and where the body portion 501 formed of elastomer (or an elastomer-based fabric).

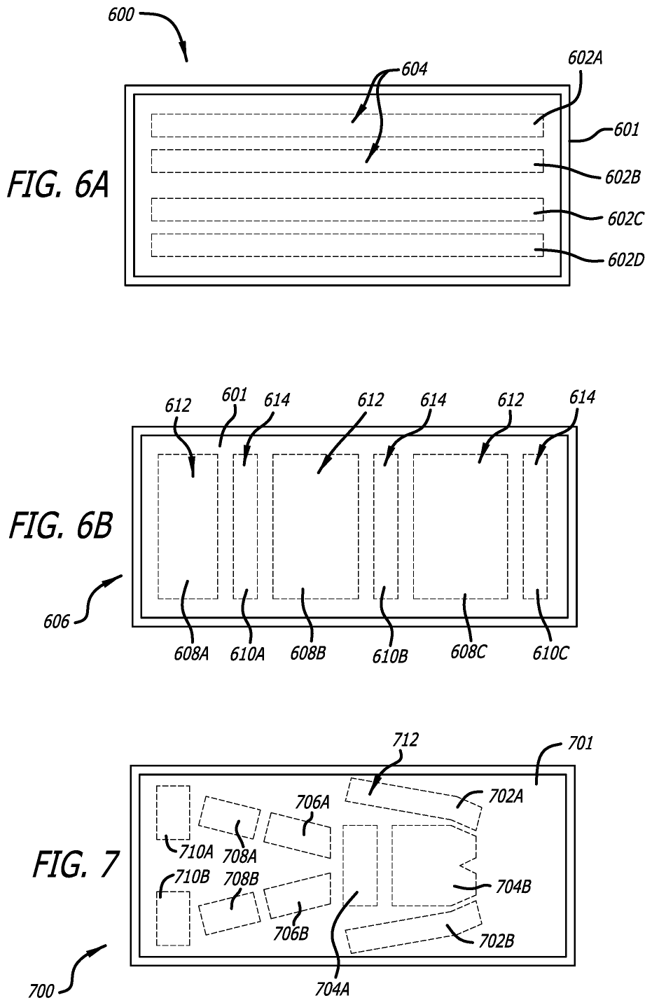

FIG. 6A is an illustration of a blanket having gel packets disposed as strips along the length of the blanket, in accordance with some embodiments. The blanket 600 is shown to be formed of an elastomer fabric 601 and include a plurality of gel pockets 602A-602D (individually and collectively “602”) that are filled with gel 604. The gel pockets 602 in FIG. 6A are disposed in an illustrative configuration in strips that run a majority (or more specifically, substantially an entirety) of a length of the blanket 600. It should be understood that the number of gel pockets 602 included may vary as may the width of the gel pockets 602.

FIG. 6B is an illustration of a blanket having gel pockets disposed as strips along the width of the blanket, in accordance with some embodiments. The blanket 606 is shown in be formed of an elastomer fabric 601 and include a first plurality of gel pockets 612 and a second plurality of gel pockets 614, with each of the gel pockets 612, 614 having a width that extends a majority (or more specifically, substantially an entirety) of a width of the blanket 606. The gel pockets 612 may have a first width and the gel pockets 614 may have a second width, the second width being different than the first width (e.g., as shown, less than the first width).

FIG. 7 is an illustration of a blanket having gel pockets disposed in the formation of a human body, in accordance with some embodiments. The blanket 700 is shown in be formed of an elastomer fabric 701 and include a plurality of gel pockets 702A-710B, each filled with gel 712, where the gel pockets 702A-710B are arranged in a configuration resembling a patient body. Such an arrangement provides for the gel pockets 702A-710B to provide weight on top of areas that are likely to be in contact with (e.g., placed on top of) the pads 120 (e.g., see FIG. 1). For instance, the blanket includes arm gel pockets 702A-702B, lower and upper torso gel pockets 704A-704B, upper leg gel pockets 706A-706B, lower leg gel pockets 708A-708B, and feet gel pockets 710A-710B.

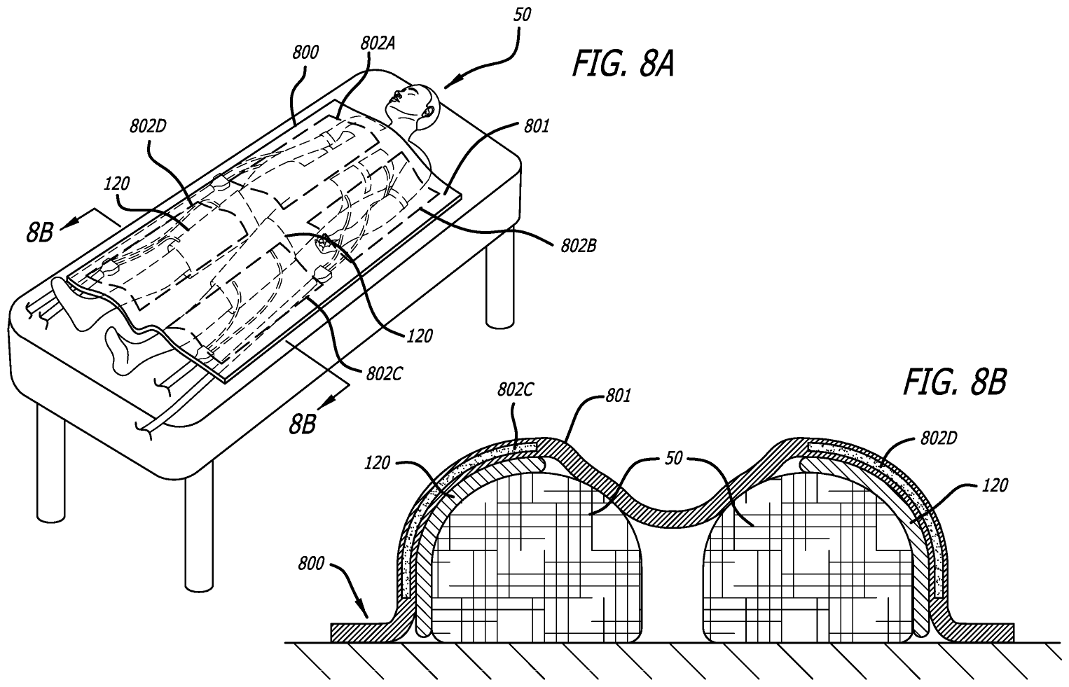

FIG. 8A is an illustration of a blanket of any embodiment disclosed herein placed on a patient body showing how the blanket contours to the patient body, in accordance with some embodiments. FIG. 8A illustrates the patient 50 on a surface (e.g., a table) with two pads 120 disposed on the legs of the patient 50. Additionally, FIG. 8A illustrates a blanket 800 placed on top of the patient 50 and the pads 120. Importantly, the blanket 800 is formed of a fabric 801 (e.g., elastomer or elastomer-based) and includes a plurality of gel pockets 802A-802D (collectively and individually, “802”). The gel pockets 802 in FIG. 8A are intended to provide an illustration of one configuration of the gel pockets 802, and as depicted in other figures as well as described herein, various configurations have been contemplated and are within the scope of this disclosure. In FIG. 8A, the blanket 800 is placed on the patient 50 and the configuration of the gel pockets 802C-802D is such that the gel pockets 802C-802D are disposed on top of the pads 120. Thus, the gel pockets 802C-802D provide weight that holds the pads 120 in place (e.g., in a stationary position) during the TTM procedure.

FIG. 8B provides a cross-sectional view of the blanket of FIG. 8A, in accordance with some embodiments. The cross-sectional view across 8B-8B as noted in FIG. 8A illustrates the legs of the patient 50 in direct contact with a bottom side of the pads 120 and the blanket 800 placed on top of the pads 120. As seen, a portion of the blanket 800 is in direct contact with an upper side of the pads 120 and a portion of the blanket 800 is in direct contact with the legs of the patient 50. Additionally, FIG. 8B illustrates that the gel pockets 802C-802D are disposed on top of the pads 120 thereby providing weight to maintain the placement of the pads 120 on the legs of the patient 50.

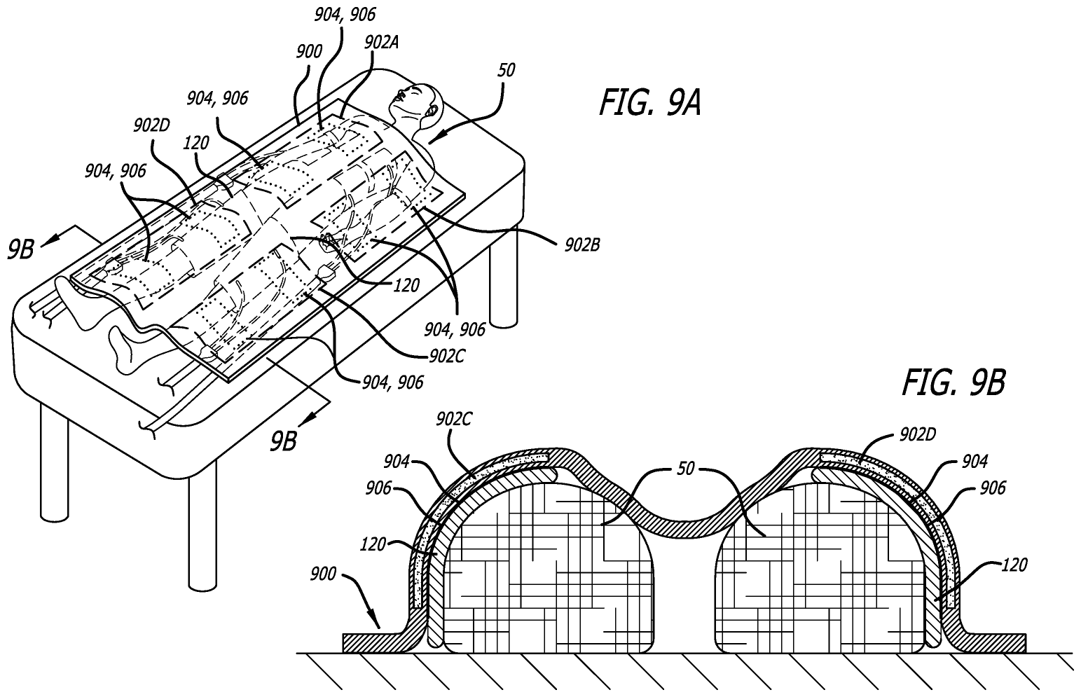

FIG. 9A is an illustration of a blanket having gel pockets and fixedly coupled to a thermal contact pad through hook and loop fasteners, in accordance with some embodiments. FIG. 9A illustrates the patient 50 on a surface (e.g., a table) with two pads 120 disposed on the torso of the patient 50 and two pads 120 disposed on the legs of the patient 50. Additionally, FIG. 9A illustrates a blanket 900 placed on top of the patient 50 and the pads 120. Importantly, the blanket 900 is formed of a fabric 801 (e.g., elastomer or elastomer-based) and includes a plurality of gel pockets 902A-902D (collectively and individually, “902”). The gel pockets 902 in FIG. 9A are intended to provide an illustration of one configuration of the gel pockets 902, and as depicted in other figures as well as described herein, various configurations have been contemplated and are within the scope of this disclosure. In FIG. 9A, the blanket 900 is placed on the patient 50 and the configuration of the gel pockets 902A-902D is such that the gel pockets 902A-902B are disposed on top of the pads 120 placed on the torso of the patient 50 and the gel pockets 902C-902D are disposed on top of the pads 120. Thus, the gel pockets 902C-902D provide weight that holds the pads 120 in place (e.g., in a stationary position) during the TTM procedure.

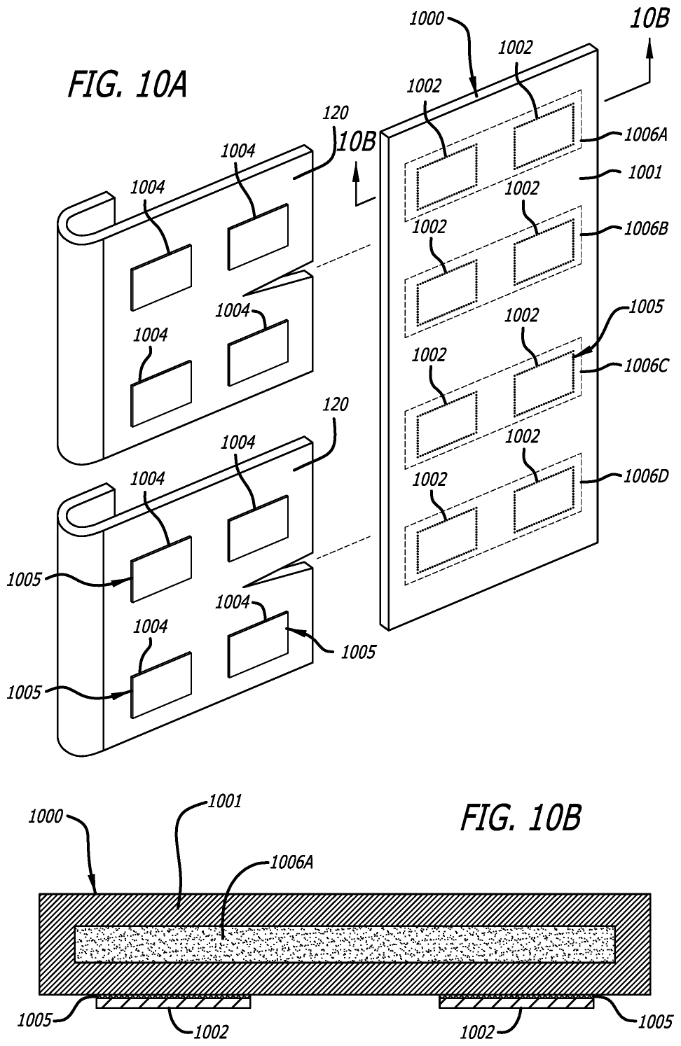

Additionally, FIG. 9A illustrates that the blanket 900 includes a plurality of coupling components 904. As shown, the blanket 900 includes eight coupling components 904, where two coupling components 904 are located in accordance with each of the gel pockets 902 (e.g., two coupling components 904 are located are on the underside of the blanket 900 in alignment with each the gel pocket, see FIG. 8B). In some embodiments, the coupling components 904 include one side of a hook and loop fastener assembly (e.g., two examples of hook and loop fastener assemblies are those often referred to as VELCRO® or DURAGRIP®). As is seen in FIG. 9B, the pads 120 may include the (opposite) corresponding side of the hook and loop fastener assembly so that the direct contact between the pads 120 and the blanket 900 may result in coupling via the hook and loop fastener assembly. In some embodiments, the hook and loop fastener assembly may be sewn each of the blanket 900 and the pads 120 or, alternatively, may be attached via an adhesive (the adhesive is shown in FIG. 10A).

FIG. 9B provides a cross-sectional view of the blanket of FIG. 9A, in accordance with some embodiments. The cross-sectional view across 9B-9B as noted in FIG. 9A illustrates the legs of the patient 50 in direct contact with a bottom side of the pads 120 and the blanket 900 placed on top of the pads 120. As seen, a portion of the blanket 900 is in direct contact with an upper side of the pads 120 and a portion of the blanket 800 is in direct contact with the legs of the patient 50. Additionally, FIG. 9B illustrates that the gel pockets 902C-902D are disposed on top of the pads 120 thereby providing weight to maintain the placement of the pads 120 on the legs of the patient 50.

Further, in the embodiment of FIGS. 9A-9B, the blanket 800 and the pads 120 are coupled using hook and loop fastener assembly. More particularly, portions of the underside of the blanket 800 include a first side of the hook and loop fastener assembly (e.g., either the hooks or the loops) and the upper side of the pads 120 include a second side of the hook and loop fastener assembly (e.g., the opposing side of the hook and loop fastener assembly that is included on the blanket 800). The inclusion of the hook and loop fastener assembly on the blanket and the pads provides additional force to maintain placement of the pads 120. in addition to the weight provided by the gel pockets 902C-902D.

FIG. 10A is an illustration of a blanket having gel pockets and magnetically coupled to a thermal contact pad, in accordance with some embodiments. FIG. 10A illustrates the coupling of a blanket 1000 and two pads 120 in an exploded view. The blanket 1000 is shown to include a plurality of gel pockets 1006A-1006D and a plurality of magnetic components 1002. The pads 120 are shown to include a plurality of magnetic components 1004 that may correspond to and align with the magnetic components 1002. Thus, when the blanket 1000 is placed on top of the pads 120 (e.g., see FIGS. 5A, 8A, and 9A), the magnetic components 1002, 1004 couple thereby providing additional force to maintain placement of the pads 120, in addition to the weight provided by the gel pockets 1006A-1006D. In some embodiments, the magnetic components 1002, 1004 may be sewn into each of the blanket 900 and the pads 120 or, alternatively, may be attached via an adhesive 1005. In some embodiments, fabric 1001 may cover the magnetic components 1002.

FIG. 10B provides a cross-sectional view of the blanket of FIG. 10A, in accordance with some embodiments. The cross-sectional view across 10B-10B as noted in FIG. 10A illustrates the blanket 1000, where the blanket 1000 is formed of a fabric 1001 (e.g., elastomer or elastomer-based) and includes gel pockets 1006A-1006D (each encompassed and closed by the fabric 1001). Although the gel pockets 1006A-1006D are illustrated as having a rectangular cross-section, the gel pockets 1006A-1006D may have alternative cross-section shapes such as square, oval, circular, etc. Additionally, the cross-sectional view of FIG. 10B illustrates that the blanket 1000 includes the magnetic components 1002 disposed on an underside of the blanket 1000. As illustrated in FIG. 10A, the magnetic components 1002 are disposed in a configuration so as to couple with the magnetic components 1004 of the pads 120.

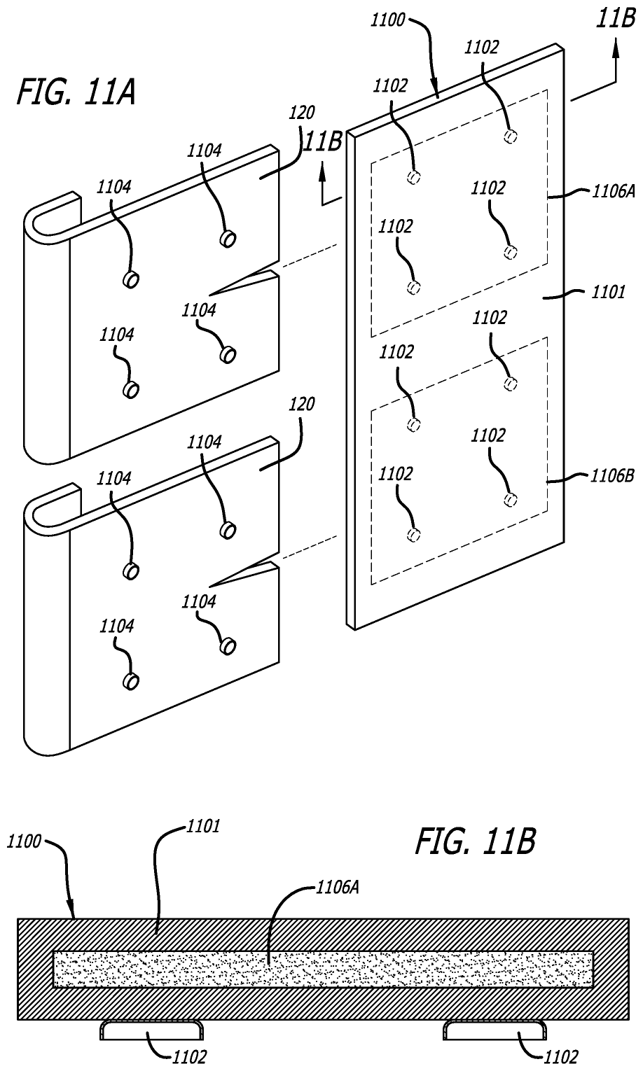

FIG. 11A is an illustration of a blanket having gel pockets and coupled to a thermal contact pad through snap fasteners, in accordance with some embodiments. FIG. 11A illustrates the coupling of a blanket 1100 and two pads 120 in an exploded view. The blanket 1100 is shown to include a plurality of gel pockets 1106A-1106B and a plurality of snap fastener components 1102. The pads 120 are shown to include a plurality of snap fastener components 1104 that may correspond to and align with the snap fastener components 1102. Thus, when the blanket 1100 is placed on top of the pads 120 (e.g., see FIGS. 5A, 8A, and 9A), the snap fastener components 1102, 1104 couple thereby providing additional force to maintain placement of the pads 120, in addition to the weight provided by the gel pockets 1106A-1106B. In some embodiments, the snap fastener components 1102, 1104 may be sewn into each of the blanket 900 and the pads 120 or, alternatively, may be attached via an adhesive. In some embodiments, fabric 1101 may cover the snap fastener components 1102.

FIG. 11B provides a cross-sectional view of the blanket of FIG. 11A, in accordance with some embodiments. The cross-sectional view across 11B-11B as noted in FIG. 11A illustrates the blanket 1100, where the blanket 1100 is formed of a fabric 1101 (e.g., elastomer or elastomer-based) and includes gel pockets 1106A-1106B (each encompassed and closed by the fabric 1101). Although the gel pockets 1106A-1106B are illustrated as having a rectangular cross-section, the gel pockets 1106A-1106B may have alternative cross-section shapes such as square, oval, circular, etc. Additionally, the cross-sectional view of FIG. 11B illustrates that the blanket 1100 includes the snap fastener components 1102 disposed on an underside of the blanket 1100. As illustrated in FIG. 11A, the snap fastener components 1002 are disposed in a configuration so as to couple with the snap fastener components 1104 of the pads 120.

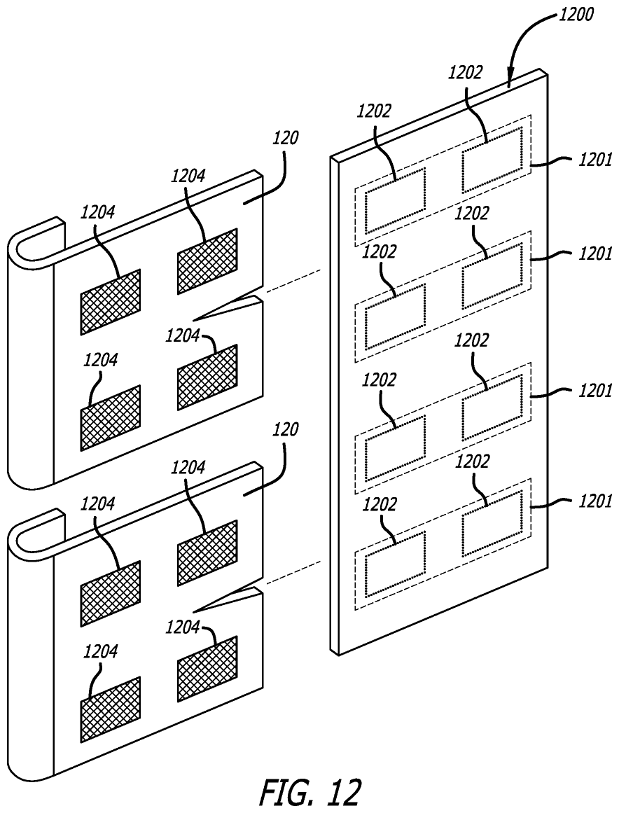

Referring to FIG. 12, an illustration of a blanket having gel pockets and adhesively coupled to a thermal contact pad is shown in accordance with some embodiments. FIG. 12 illustrates the coupling of a blanket 1200 and two pads 120 in an exploded view. The blanket 1200 is shown to include a plurality of gel pockets 1201. Additionally, FIG. 12 illustrates that the blanket 1200 and/or the pads 120 may include one or more adhesive patches 1202, 1204 disposed thereon. Thus, when the blanket 1200 is placed on top of the pads 120 (e.g., see FIGS. 5A, 8A, and 9A), the adhesive components 1002, 1004 create an adhesive coupling between the blanket 1200 and the pads 120 thereby providing additional force to maintain placement of the pads 120, in addition to the weight provided by the gel pockets 1201.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A blanket comprising:
a body portion formed of fabric, wherein the fabric is elastomer or elastomer-based; and
a first pocket within the body portion and surrounded by the fabric, wherein the first pocket is an enclosed area filled with an insulative material, and wherein the blanket is configured to be placed on at least a first thermal pad of a targeted temperature management (TTM) system, wherein the first pocket is configured to provide a weight on top of the first thermal pad.

2. The blanket of claim 1, wherein the insulative material is an insulative gel.

3. The blanket of claim 1, wherein a length of the first pocket is substantially an entirety of a length of the blanket.

4. The blanket of claim 1, wherein a width of the first pocket is substantially an entirety of a width of the blanket.

5. The blanket of claim 1, further comprising:
a plurality of pockets within the body portion, wherein the plurality of pockets includes the first pocket and wherein each of the plurality of pockets is surrounded by the fabric, and is an enclosed area filled with the insulative material.

6. The blanket of claim 5, wherein each of the plurality of pockets has a length that is substantially an entirety of a length of the blanket, and the plurality of pockets are aligned in a side-by-side configuration.

7. The blanket of claim 5, wherein each of the plurality of pockets has a width that is substantially an entirety of a width of the blanket, and the plurality of pockets are aligned in a side-by-side configuration.

8. The blanket of claim 1, wherein the blanket includes a coupling component configured to couple with the first thermal pad.

9. The blanket of claim 8, wherein the coupling component is one of an adhesive patch, a snap fastener component, a hook and loop fastener component, or a magnetic component.

10. The blanket of claim 9, wherein when the coupling component is any of the snap fastener component, the hook and loop fastener component, or the magnetic component, the coupling component is configured to couple with a reciprocal coupling component of the first thermal pad.

11. A targeted temperature management (TTM) system, comprising:
a TTM module configured to provide a TTM fluid;
a thermal pad configured to receive the TTM fluid from the TTM module to facilitate thermal energy transfer between the TTM fluid and a patient;
a fluid delivery line (FDL) extending between the TTM module and the thermal pad, the FDL configured to provide TTM fluid flow between the TTM module and the thermal pad; and
a blanket comprising:
a body portion formed of fabric, wherein the fabric is elastomer or elastomer-based, and
a first pocket within the body portion and surrounded by the fabric, wherein the first pocket is an enclosed area filled with an insulative material, and wherein the blanket is configured to be placed on at least the thermal pad of a targeted temperature management (TTM) system, wherein the first pocket is configured to provide a weight on top of the thermal pad.

12. The TTM system of claim 11, wherein the insulative material is an insulative gel.

13. The TTM system of claim 11, wherein a length of the first pocket is substantially an entirety of a length of the blanket.

14. The TTM system of claim 11, wherein a width of the first pocket is substantially an entirety of a width of the blanket.

15. The TTM system of claim 11, wherein the blanket further comprises a plurality of pockets within the body portion, wherein the plurality of pockets includes the first pocket and wherein each of the plurality of pockets is surrounded by the fabric, and is an enclosed area filled with the insulative material.

16. The TTM system of claim 15, wherein each of the plurality of pockets has a length that is substantially an entirety of a length of the blanket, and the plurality of pockets are aligned in a side-by-side configuration.

17. The TTM system of claim 15, wherein each of the plurality of pockets has a width that is substantially an entirety of a width of the blanket, and the plurality of pockets are aligned in a side-by-side configuration.

18. The TTM system of claim 11, wherein the blanket includes a coupling component configured to couple with the thermal pad.

19. The TTM system of claim 18, wherein the coupling component is one of an adhesive patch, a snap fastener component, a hook and loop fastener component, or a magnetic component.

20. The TTM system of claim 19, wherein when the coupling component is any of the snap fastener component, the hook and loop fastener component, or the magnetic component, the coupling component is configured to couple with a reciprocal coupling component of the thermal pad.

21-30. (canceled)
